(19) United States
(12) Patent Application Publication
(10) Pub. No.: US 2004/0230228 A1
(43) Pub. Date: Nov. 18, 2004

(54) Device for treatment

(76) Inventor: Tatsuzo Nakamura, Hyogo (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/782,828

(22) Filed: Feb. 23, 2004

(30) Foreign Application Priority Data
Feb. 24, 2003 (JP) JP2003-096556

(51) Int. Cl. A61N 1/18

(52) U.S. Cl. 607/3

(57) ABSTRACT

A therapeutic instrument, which is usable to apply, accordingly to the choice of the user, mechanical stimuli alone or electric stimuli together with mechanical stimuli individually to each of a plurality of points on the body surface related to treating somatic disorders, is being disclosed. The said therapeutic instrument is characterized as being comprised of supporting medium for wearing it on body and conductive projections lined on the inner surface of the said supporting medium to apply pressure on multiple regions of body surface where the said projection has a hole on the bottom face, which accepts an electrode terminal of compatible shape and size. The said therapeutic instrument allows a non-expert user to accurately apply pressure stimuli alone or electric stimuli concurrently with the pressure stimuli accordingly to his/her choice.

### TECHNICAL FIELD

The invention relates to a therapeutic instrument to be utilized for physical therapies to remedy abnormal human body conditions or somatic symptoms.

### BACKGROUND ART

In the endeavor to treat various conditions, maladies, and somatic disorders as well as symptoms arising therefrom, application of stimuli such as those evoked with electricity, magnetism, heat, and acoustic waves as well as pressing down or pulling by mechanical forces have been widely adapted as therapeutic techniques. However, there has not been a known convenient therapeutic instrument that allows non-expert users to apply, at time and place of their choice, stimuli evoked by physical pressure alone or concurrently by both physical pressure and electricity to so-called acupressure points based on their subjective symptoms.

### DISCLOSURE OF INVENTION

### The Problem to be Solved by the Invention

Based on the above-mentioned background, the invention has a purpose of providing the therapeutic instrument which allows its users to apply physical pressure or electric stimuli concurrently with such physical pressure to meridian points or also known as pressure points.

### Means to Solve the Problem

To fulfill such purpose, the inventor has completed the invention as a result of multiple studies in search of a therapeutic instrument, which is capable of concurrently applying mechanical stimuli preferably to three or more regions of a human body and, depending of its user's choice, is also capable of easily applying electric stimuli concurrently with the mechanical stimuli to each of the regions, furthermore, allows its user to wear it all the while he/she conducts activities of daily living in order to receive mechanical stimuli on acupressure points.

More specifically, the invention provides the following.

(1) A therapeutic instrument, which is characterized in that it is usable to apply, accordingly to the choice of the user, mechanical stimuli alone or electric stimuli concurrently with mechanical stimuli individually to each of the plurality of points on the body surface related to treating somatic disorders, comprising of supporting medium for wearing it on body and conductive protrusions lined on the inner surface of the said supporting medium to apply pressure on multiple regions of body surface where the said projection has a hole on the bottom face which accepts an electrode terminal of compatible shape and size.

(2) The therapeutic instrument of the above 1, wherein the said mechanical stimuli is pressure to body skin surface.

(3) The therapeutic instrument of the above 1 or 2, wherein the said electric stimuli are of low frequency or high frequency.

(4) The therapeutic instrument of any of the above 1 to 3, wherein the hole on the bottom face of the said protrusion has larger diameter at its deeper portion than at the entry of the hole.

(5) The therapeutic instrument of any of the above 1 to 4, wherein the said protrusion constitutes of a convex part and a base part which are connected to each other, holding the supporting material in between them.

(6) The therapeutic instrument of one of claims 1 to 5 which is characterized by the said supporting material being bands, supporters, body suites, eyeglass frames, goggle frames, or circum-ocular pads.

### Effect of the Invention

The above invention allows its user to individually apply mechanical stimuli or mechanical stimuli concurrently with electric stimuli to each of the acupressure points according to his/her own choice. Also, since there is no outward projection on the protrusion, the invention enables the user to wear it in order to continuously apply mechanical stimuli to acupressure points while he/she conducts activities of daily living, furthermore it allows the user to easily connect electrode terminals of high (low) frequency therapeutic instrument in order to additionally apply electric stimuli when the user wishes to do so.

### Best Mode for Carrying Out the Invention

In regard to the invention, “a plurality of points on the body surface related to treating somatic disorders” refers to a plurality of meridian points (hereinafter referred to as ‘acupressure points’) known in theories and practice of oriental medicine or any other points that are capable of causing idiosyncratic somatic reactions to physical stimuli applied. In addition, in regard to the invention, “treatment” refers to a concept which not only includes treatment of specified diseases, but also relieving one's fatigue, improving physical conditions, and maintaining or improving one's health.

In regard to the invention, “pressure” means topically or locally pressing down. Although it is not possible to define what is topical or local to exact certainty, it generally refers to an area, for instance, where diameter is 1 to 10 mm.

In regard to the invention, “supporting material” includes any items such as articles of clothing (such as underwear, pants, body suits), bands (such as wristlets and hair bands), supporters, eyeglass frames, goggle frames, circum-ocular pads which have parts that directly come in contact with skin so that they allow attached protrusions to be continuously held against or adjacent to prescribed positions of the human body. Also, it may be an item that includes zonary reinforcements such as bands affixed to the outer or inner side of an article of clothing.

In regard to the invention, there is no limitation as to the shape of the “protrusion,” so it may be of any shape as long as is not too sharp to injure human skin surface and is suitable to apply pressure, for instance, an approximate circular cone with a rounded tip, an item constituting of a paraboloid, or frustum of circular cone or a frustum of pyramid wherein the width of the tip is approximately 1 mm. Also, a plurality (e.g., 2 to 7) of small protrusions of the same shape may be protruding from a base material to form a unit as a whole. Also, the protrusions can be placed on a supporting material so that they come in contact with all or some of acupressure points intended for stimulation that are located in each of the regions of the body where supporting material is to be worn, and location of such acupressure points are well known to persons skilled in the art.

The above-mentioned protrusion is produced of conductive material. Typical example of conductive material would be, but not limited to, metal, conductive rubber or plastic with added ingredient such as carbon, or bridged aqueous acrylic resin. The hole equipped on the bottom face of the protrusion for the invention is designed to accommodate an electrode terminal to be inlet from high frequency or low frequency therapy device(s). By having the electrode terminal shaped and sized to be compatible with the said hole, one can inlet the electrode terminal from the outer side of the supporting material into the hole on the bottom face of the protrusion to have the protrusion carry electric current. Since the protrusions are configured in this manner, when required stimuli is pressure and not of electricity, there will be no outward projections, for example, of electrodes from the protrusions so that there is no hindrance in wearing the supporting material while an user conducts activities of daily living. Commonly known and available high frequency of low frequency therapy device of any kind can be utilized, by only adapting the shape of electrodes to be compatible with the holes on the protrusions, to apply frequency ranging from 1 to several tens of thousand hertz. Also, the voltage can be the same as those commonly known and available therapeutic instruments, and can be selected within a range of a few volts to several hundred volts, depending on symptoms and frequencies.

The method in which protrusions are attached to a supporting material is not confined to any particular one. For instance, protrusions can be joined with the surface of a supporting material with an adhesive agent applied on the circumference of the holes of the bottom faces of the protrusions. Alternatively, a protrusion can be constituted from two parts, a convex part and a base part, and these two parts can be joined together holding a supporting material in between them. In such a case, ad libitum method to join the two parts can be deployed. For example, the convex part may have a hole on its bottom face and the surface of the base part may be projected so that pressing the projection into the hole of the convex part can fixate it, or one or more parts to accept mutual intrusions may be placed in between the convex and base parts; means such as these allow the two parts to be fixated together while they hold supporting material in between them. An adhesive agent may be applied to the part that accepts mutual intrusions by the two parts. In case where the protrusion is consisted of a convex part and a base part, the base part would have a hole on its bottom face to accept the said electrode terminal. In case where the bottom face of the protrusion is joined with the inner surface of the supporting material by such means as an adhesive agent, any appropriate means, such as punching holes on the supporting material, can be deployed to allow an electrode terminal to be inserted into the said hole on the bottom face of the said protrusion. In case where the protrusion is consisted of a convex part and a base part, the supporting material may have holes at the corresponding places so that the two parts of the protrusion can be joined together.

It is desirable that the hole, which is made on the bottom face of the protrusion to accommodate an electrode terminal, is structured so that it is able to provide detachable connection (for example, refer to FIG. 3) like a snap faster (or a press fastener). More specifically, the hole for accommodating the electrode terminal can be made to have a wider diameter at its deeper part than it does at the entry of the hole. Correspondingly to the form of the hole, the electrode terminal can be shaped to have a bulge at its tip in order to enable easy connection and disconnection as required. In a case where the protrusion is formed of an elastic body such as conductive rubber, then the electrode terminal to be accommodated in to the hole thereof may be made of a hard material such as a metal, and in another case where the protrusion is made of a metal, the electrode terminal may be made to be elastic. Also, even when the protrusion is made of a metal, if the inner side of the hole thereof is structured to achieve easy elastic deformation, electrode terminal made of metal may be used. It is desirable to shape the electrode into that of, for instance, a male snap fastener so that the electrode terminal does not outwardly project when carrying electric current.

The following description is considered as illustrative only of the typical embodiments of the invention, it is not the inventor's intention to limit the invention to the exact construction and operation shown and described herein.

### EXAMPLE 1

FIG. 1 depicts a side plan view of an example of the protrusion as a component part of the invention. Protrusion 1 is made of conductive rubber or a metal, and the diameter of the bottom face 2 is 7.5 mm and the length from the bottom face 2 to the apex is 5 mm; however, the size dimensions may be increased or decreased accordingly to fit the region of the body whereon it is to be worn, for instance, the diameter of the bottom face may be convenient and effective when it is between 3 to 10 mm and the length from the bottom face to the apex between 3 to 7 mm. The figure depicts the bottom face as circular in form, but it is not limited to be so; it may be of other appropriate shapes such as an ellipse and a polygon. The contacting surface 4 to the body surface on the protrusion 1 is an approximate paraboloid, but it may be a circular cone, spherical surface, or any other appropriate convex surface.

In the FIG. 1, 5 depicts the hole on the bottom face 2 where the entry part is relatively narrow and its deep part has a wider diameter than the entry part. The protrusion 1 is affixed at the bottom face 2 to the inner surface of a supporting material of suitable shape, corresponding to the region of the body whereon it is to be worn. The fixation, for instance, may be achieved by joining the circumference of the hole 5 to the bottom face 2 with an adhesive.

### EXAMPLE 2

FIG. 2 depicts the protrusion 11 of the example 2. This protrusion 11 consists of a plurality (3 in the Figure) of small protrusions to contact the body surface, 14a, 14b, and 14c, which are projected from the discoid base material 12. The base material 12 has the hole 15, which has the same characteristics as the hole 5 in the example 1. The fixation of protrusion 11 to a supporting material is achieved in the same manner as described in the example 1.

FIG. 3 depicts the illustration of the protrusion 21 and the electrode 28, which carries the output electricity to the protrusion via a conducting wire 27 extending from a high (low) frequency therapy device, prior to being connected to each other. Depicted in the figure is the electrode terminal 26 instituted on one side of the electrode 28, and it is of a shape that corresponds to the hole 25 on the bottom face of the protrusion 21 to be inserted into it in a snap-fastener like manner. The electrode terminal 26 is inserted (via an opening instituted as required on the supporting material) into the hole 25 of the protrusion 21 fixed on any appropriate supporting material to achieve engaging of these parts in a detachable manner just like a snap fastener.

### EXAMPLE 3

FIG. 4 depicts the illustration of the protrusion 31 of the example 3, which constitute of 2 parts, and the electrode 38, which carries the output electricity to the protrusion via a conducting wire 37 extending from a high (low) frequency therapy device, prior to being connected to each other. In this example, the protrusion 31 constitutes of the convex part 32 and the base part 33. On the bottom face of the convex part 32 is a hole 34, and on the corresponding face of the base part 33 has a projection 39 of compatible shape with the hole 34; their construction allows them to be joined into a unity by pushing the projection 39 into the hole 34. The convex part 32 and the base part 33 can be fixed on the supporting material by fitting the projection 39 into the hole 32 via an opening instituted on the supporting material in order for letting the projection 39 through. Also the base part 33 of the protrusion 31 has a hole 35. The hole 35 has a wider diameter at its deep part than at the entry, and it is constructed to accommodate, in a detachable manner, electrode terminal 36 of a shape corresponding to the electrode 38.

### EXAMPLE 4

FIGS. 5 and 6 depict an illustration of a case where a plurality of electrodes 8 is fixated to a plurality of protrusions 1. FIG. 5 depicts a case where a plurality of the protrusions concurrently carry current via a single conducting wire, and FIG. 6 depicts a case where the current is carried to each of the protrusions via an individual conducting wire. In the latter case, electric stimuli can be independently applied to each of the protrusions 1.

FIG. 7 depicts a plurality of protrusions 1 attached to the supporting material 19.

### EXAMPLE 5

FIG. 8 depicts an example of the invention where it is formed into a hair band. In the figure, the body of the hair band 40, the supporting material, may be made of material common to ordinary hair bands intended to closely contact the sincipital region; a plurality of the protrusions 41 are attached to a plurality of locations on the hair band so that the convex parts face the scalp. The uppermost protrusion 41 within the figure is adjusted with the cord 43 to be aligned on the vertical meridian going through the pupillary center. An elastic material such as a rubber band connects the lower ends of the hair band so that it is compatible with a sincipital region of varying sizes. The electrodes 48 are not connected to each of the protrusions 41 in the figure, and it is depicting that the mechanical stimuli (pressure) alone caused by the protrusions are being applied to the sincipital region. The electrodes 48 can apply electric stimuli when the user mounts the electrode terminals 45 to any of the plurality of the protrusions 41 as he/she wishes and turns on electricity from the high (low) frequency therapy device 49.

### EXAMPLE 6

FIG. 9 depicts an illustration of an example of the invention as a knee supporter. The upper half depicts the invention for a right knee and the lower half a left knee. In the figure the body of the knee supporter is depicted as 50 whereon the protrusions 51 are inwardly attached. The item 53 refers to the belt affixed on the surface of the body of the knee supporter. The belt 53 is useful in enhancing the pressure applied by the protrusions 51. The protrusions 51 are attached to the body of the knee supporter in the manner described in the above example 3.

### EXAMPLE 7

FIG. 10 depicts the elevational view of the body of the knee supporter 50 of the example 6 wherein the zippers 55 are incorporated on its centerline on the anterior surface to allow zipping, unzipping, and undoing a zip. Three sets of the zippers 55 are incorporated on the right and left halves of the body of the knee supporter, and are useful in enabling adjustment of the diameter of the supporter to be compatible with the girth of the user's knee without causing the central axis of the knee supporter to be out of alignment by using outmost, middle, or innermost zipper depending on the girth of the user's knee.

### EXAMPLE 8

FIGS. 11 and 12 depicts an example of the invention as formed into the body suit 60. FIG. 11 depicts the frontal view of the body and the FIG. 12 depicts the back view of the body. In the FIG. 12, W indicates the distance of the protrusions 61 from the median line, which is generally 3.5 to 4 cm. In the figure, the protrusions 61 are attached inwardly to the body suit 60 directly and on locations reinforced with the belt 63. The belt 63 can be of any material as long as it has enough strength to support the protrusions 61. According to this example, for instance, a user can apply just the mechanical stimuli without electric current during the day to stimulate pressure points, and in such time as evening he/she can insert the electrode terminals to any of the protrusions 61 located at the region he/she wishes to apply electric stimuli. Also, the belt 63, for instance, may be a conductive belt woven of fibers with added silver, and it may be affixed to the outer surface of the body suit whereon attached protrusions are inwardly projecting from the body suit surface. In such a case, connecting an electrode terminal to one of the protrusions 61 and supplying electric current thereto will enable the current to be concurrently carried to a group of the protrusions located on the conductive belt with electrical connection so that the group of the protrusions can concurrently apply electric stimuli to the body.

### EXAMPLE 9

FIG. 13 depicts the example 9 of the invention formed into a circum-ocular pad. In this example, the circum-ocular pad 70 has conductive protrusions 71a through 71d and 72a through 72d attached on the frame 73 made of thin flexible plastic, cellophane, or any other flexible material so that the protrusions face the circum-ocular skin. The protrusions 71a through 71d have a hole on their bottom faces to enable electrode terminal connected to a high (low) frequency therapy device to be inserted in a detachable manner just as the snap fastener, and the holes are exposed on the surface of the frame 73. The plurality of protrusions depicted with a circle (such as 72a through d) does not have a hole in this example; instead, they are electrically connected with any of the protrusions, which are shaped like a snap fastener. Such an electrical connection is produced with, for instance, plating such as gold plating, a thin film such as that of aluminum, a conducting wire incorporated within the frame, or any other appropriate conductive means. Alternatively, a part of the frame whereon the protrusions to be connected electrically are located may be made of a metal (e.g., copper plate) and a part of the frame located between mutually insulated protrusions may be made of an insulation material such as plastic, and any protrusions, such as 72a through 72d, that are not holed like a snap fastener may be formed by projecting metal surface thereof. As for the electrical connection, the figure conceptually depicts 4 groups of the protrusions connected with a broken line: 71a through 72a, 71b through 72b, 71c through 72c, and 71d through 72d, and each of these 4 groups of protrusions are respectively connected electrically, but they are mutually insulated in the areas of the frame located between any two among the 4 groups. The item 74 depicts the nose bridge made of a flexible insulating material such as plastic, which can be bent by hands. The circum-ocular pad 70 allows a user to attach its both ends to both sides of his/her face with, for instance, bandages and insert electric terminals extending from a high (low) frequency therapy device to holed protrusions, 71a through 71d, to individually apply electric stimuli to each of the 4 areas respectively. Also, each of the protrusions applies mechanical stimuli to pressure points when electric stimuli are not required. A therapeutic instrument whereon the protrusions are located in the same manner as this example can be structured using an eyeglass frame or a goggle frame as the supporting material.

### CLINICAL EXAMPLE 1

A hair band, which incorporated a plurality of protrusions at locations depicted by the FIG. 8, was created. The protrusions located on the sincipital region, one on the right side and the other on the left, are artfully aligned on the vertical meridian going through the pupillary center. An asthenopic patient was made to wear it and low frequency, 9 V and 15 Hz, was applied for the duration of 15 minutes, demonstrating an excellent results.

### CLINICAL EXAMPLE 2

A knee supporter was created by incorporating, on 3 separate circuits, a plurality of protrusions located in the manner depicted with the FIG. 9; and the protrusions were placed on the joint cloft of an arthralgia patient. High frequency, 15 V and 10,000 Hz, was applied to the protrusions for the duration of 15 minutes, significantly alleviating the pain.

### CLINICAL EXAMPLE 3

A body suit was created by incorporating, on 3 separate circuits, a plurality of protrusions located in the manner depicted with the FIGS. 11 and 12. A patient was made to wear it while low frequency, 6V and 3 Hz, was applied for a long time. Excellent results were shown for preventing and relieving stiff neck, lower back pain, cold constitution, and constipation.

### INDUSTRIAL APPLICABILITY

The present invention provides a therapeutic instrument which allows a non-expert user to accurately apply pressure stimuli alone or electric stimuli concurrently with the pressure stimuli accordingly to his/her choice.

### BRIEF DESCRIPTION OF DRAWINGS

FIG. 1 depicts an oblique perspective figure of the protrusion in the example 1 FIG. 2 depicts an oblique perspective figure of the protrusion in the example 2.

FIG. 3 depicts protrusions and electrodes prior to connection.

FIG. 4 depicts the protrusions of the example 3 and the electrodes prior to being connected.

FIG. 5 depicts a conceptual diagram showing a method in which electric current is carried to a plurality of protrusions.

FIG. 6 depicts yet another conceptual diagram showing a method in which electric current is carried to a plurality of protrusions.

FIG. 7 depicts a conceptual diagram showing an example in which electric current is carried to a plurality of protrusions attached to a supporting material.

FIG. 8 depicts an oblique perspective view of an embodiment of the invention in the form of a hair band of example 5.

FIG. 9 depicts views from 4 different perspectives of an embodiment of the invention in the form of a knee supporter of the example 6.

FIG. 10 depicts an elevation view of yet another embodiment of the invention in the form of a knee supporter.

FIG. 11 depicts an elevation view of an embodiment of the invention as a body suit of the example 8.

FIG. 12 depicts a back view of an embodiment of the invention as a body suit of the example 8.

FIG. 13 depicts an elevation view of an embodiment of the invention as a circum-ocular pad of the example 9.

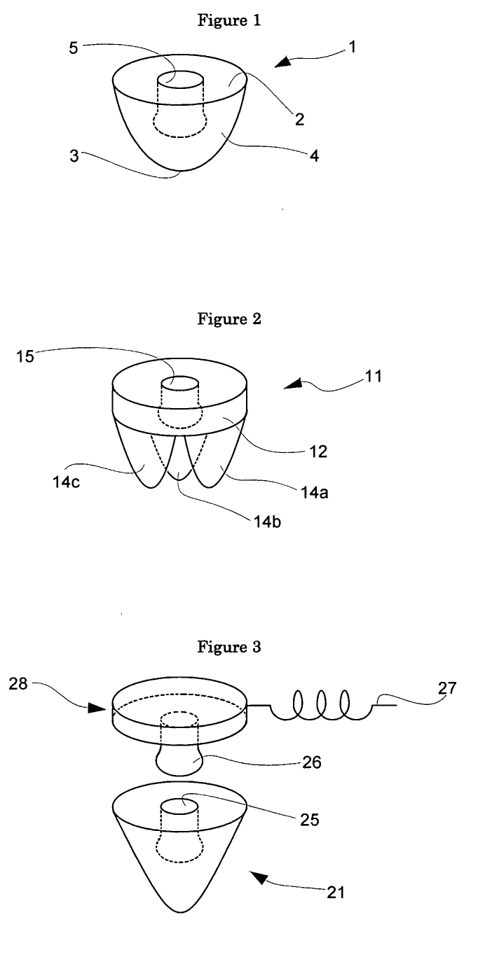

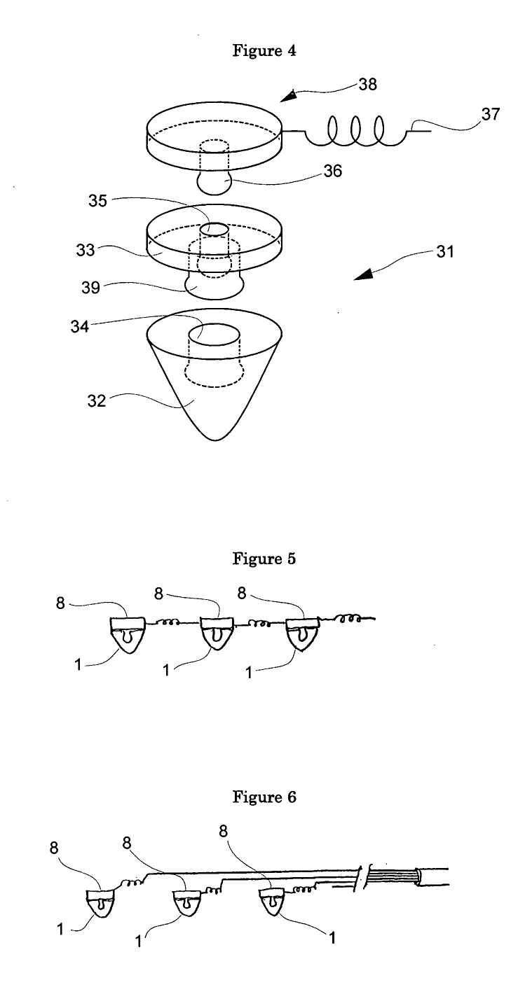

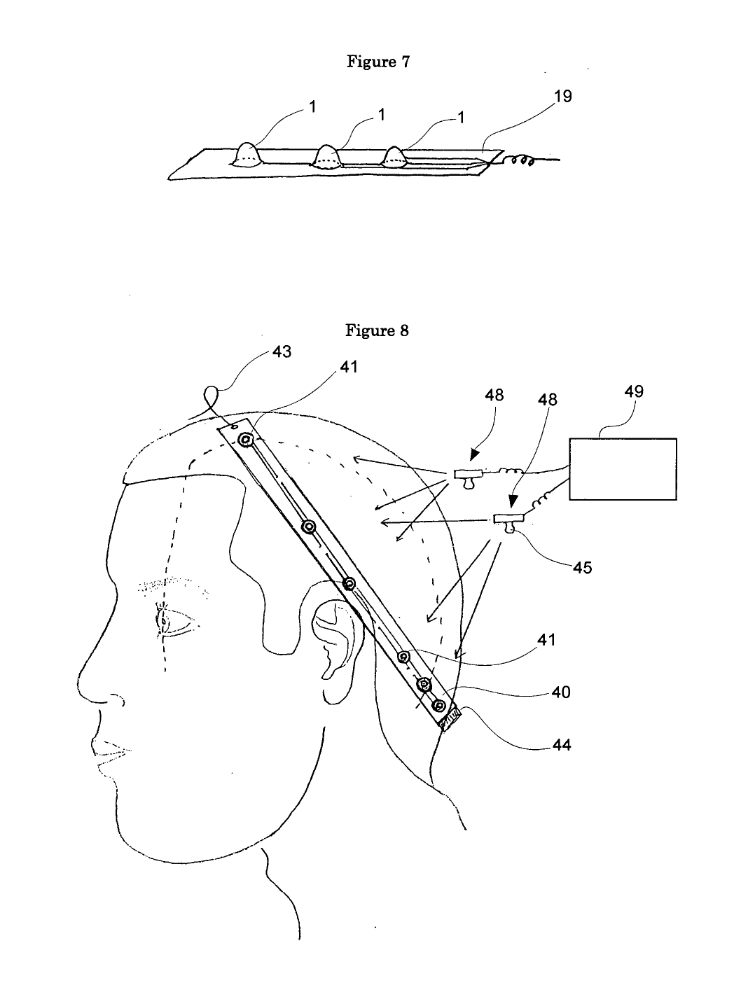

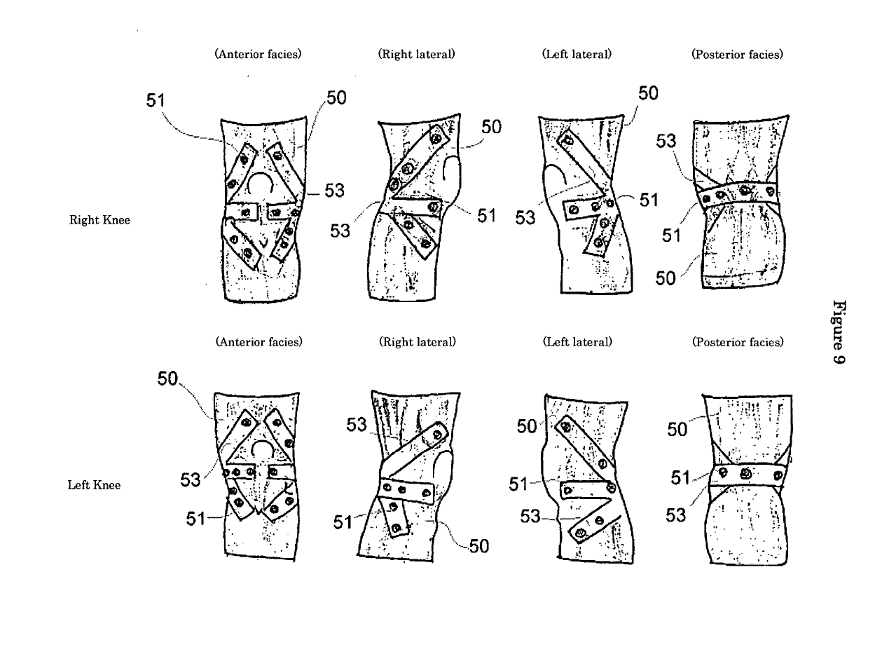

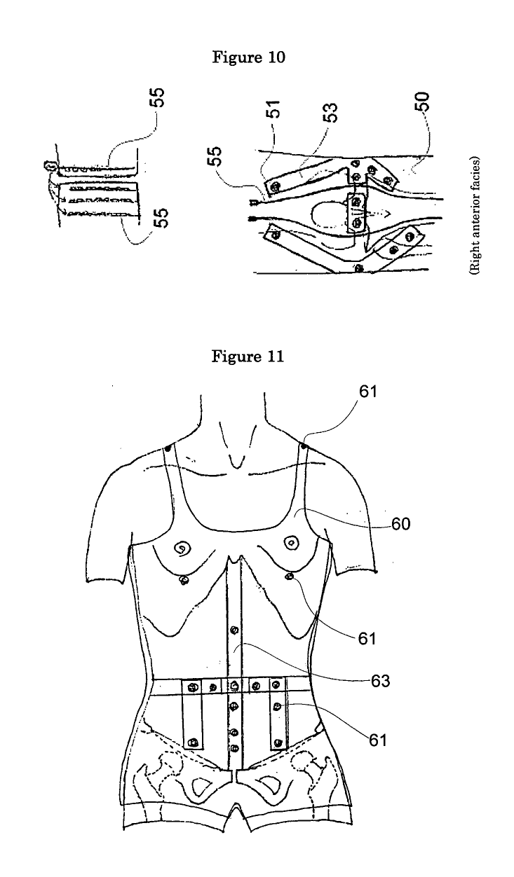

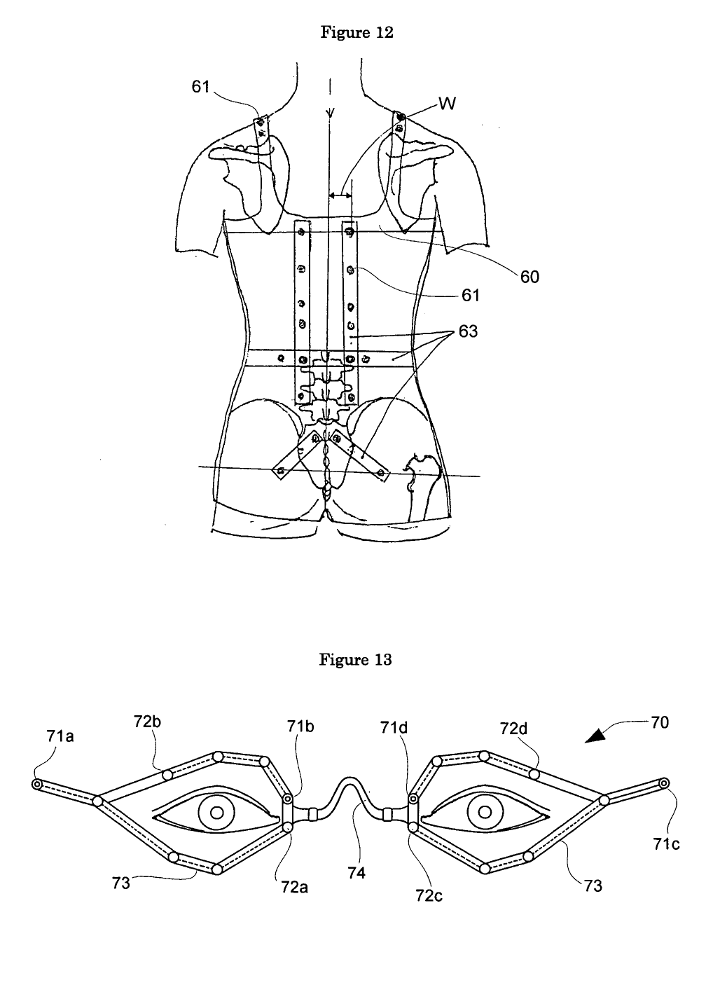

### REFERENCE NUMBERS

1: Protrusion

2: Bottom Face

3: Tip

4: Contacting surface to the body surface

5: Hole

11: Protrusion

12: Base material

14a, b, and c: Small Protrusions

15: Hole

19: Supporting Material

21: Protrusion

25: Hole

26: Electrode Terminal

27: Conducting Wire

28: Electrode

31: Protrusion

32: Convex Part

33: Base Part

34: Hole

35: Hole

38: Electrode

39: Projecting Part

40: Hair Band

41: Protrusion

43: Cord

48: Electrode

49: High (Low) Frequency Therapy Device

50: Knee Supporter

51: Protrusion

53: Belt

55: Zip Fastener

60: Body Suit

61: Protrusion

63: Belt

70: Circum-ocular Pad

71a, b, c, and d: Protrusions

72a, b, c, and d: Protrusions

73: Frame

74: Nose Bridge

1. A therapeutic instrument, which is characterized in that it is usable to apply mechanical stimuli alone or electric stimuli together with mechanical stimuli individually to each of the plurality of points on the body surface related to treating somatic disorders, it constitutes of supporting medium for wearing it on body and conductive projections lined on the inner surface of the said supporting medium to apply pressure on multiple regions of body surface where the said projection has a hole on the bottom face which accepts an electrode terminal of compatible shape and size.

2. The therapeutic instrument of the claim 1, wherein said mechanical stimuli is pressure to body skin surface.

3. The therapeutic instrument of the claim 1, wherein the said electric stimuli are of low frequency or high frequency stimuli.

4. The therapeutic instrument of the above claim 1, wherein the hole on the bottom surface of the said protrusion has larger diameter at the deep portion than at the entry of the hole.

5. The therapeutic instrument of the above claim 1, wherein the said protrusion constitutes of a convex part and a base part that are connected to each other, holding the supporting material in between them.

6. The therapeutic instrument of claim 1 which is characterized by the said supporting material being bands, supporters, body suites, spectacle frames, goggle frames, or circum-ocular pads.
